(19) United States
(12) Patent Application Publication
(10) Pub. No.: US 2021/0287275 A1
(43) Pub. Date: Sep. 16, 2021

(54) VALUE BASED COMMODITY SELECTION

(71) Applicant: GreenChoice, PBC, Somerville, MA (US)

(72) Inventors: Galen G. Karlan-Mason, Somerville, MA (US); Varun Srivastava, Somerville, MA (US)

(21) Appl. No.: 17/201,641

(22) Filed: Mar. 15, 2021

Related U.S. Application Data
(60) Provisional application No. 62/989,516, filed on Mar. 13, 2020.

(51) Int. Cl. G06Q 30/06; G06Q 30/02; G16H 20/60; G06Q 10/08; G06Q 30/00; G06Q 50/26; G06F 16/33; G06F 40/284; G06K 9/00; G06K 9/62

(52) U.S. Cl. CPC G06Q 30/0631; G06Q 30/0282; G06Q 30/0201; G16H 20/60; G06Q 10/087; A23L 33/30; G06Q 50/26; G06F 16/3331; G06F 40/284; G06K 9/00624; G06K 9/6201; G06Q 30/018

(57) ABSTRACT

A platform for GUI based consumer selection of commodity and grocery food products that align with their dietary preferences and values, with an end goal to make grocery shopping more informed and efficient. Dietary restrictions or preferences may be aligned with purchases in response to allergies, medical, or general wellness goals. Purchases consistent with core values or principles such as sustainable, ethically sourced, or unprocessed foods may be identified and filled. GUI access via a mobile device app facilitates timely validation of contemplated purchases by comparisons via a knowledge base having gathered attributes of supported products.

### RELATED APPLICATIONS

This patent application claims the benefit under 35 U.S.C. § 119(e) of U.S. Provisional Patent App. No. 62/989,516, filed Mar. 13, 2020, entitled “VALUE BASED GROCERY APPLICATION,” incorporated herein by reference in entirety.

### BACKGROUND

Selection of commodity and other retail items such as grocery and food denotes the terminus of an extended supply chain of modern mass production and distribution of consumer items. Typical consumers purchase multiple grocery items in periodic transactions of a relatively large number of items. Conventional grocery items lend little indication of their origin or distribution, and only a modicum of detail about ingredients. A typical consumer makes a purchase decision based only on a product appearance on a retail shelf, as details about farther-reaching nutrition, artificial processing, environmental implications and food safety related to the origin, manufacturing and distribution of the product remain unavailable.

### SUMMARY

A platform for GUI-based selection of consumer, commodity and grocery food products that align with dietary preferences and values, with an end goal to make grocery shopping more informed and efficient, provides a recommendation engine for rendering analytics pertaining to purchases. Dietary restrictions or preferences may be aligned with purchases in response to allergies, medical, or general wellness goals. Purchases consistent with core values or principles such as sustainable, ethically sourced, or unprocessed foods may be identified and fulfilled. GUI access via a mobile device app, or other suitable computing device, facilitates timely validation of contemplated purchases by comparisons via a knowledge base having gathered attributes of supported products.

Modern consumers are increasingly aware of the impact of their activities on an overall global system of human existence. Many individuals seek to be aware of natural and infrastructure systems and processes affecting human existence. As the dominant species on the planet, humans are uniquely qualified to both observe and change the aspects of human existence that impact the natural, economic, environmental and societal trends that have evolved.

As a result, an individual value system evolves, as each person realizes the larger effect of their existence and longevity. Many people want assurances that their lifestyle choices have a minimal detrimental effect on the overall global system. For example, a so-called “carbon footprint” has evolved as a barometer by which an impact a given entity has on overall sustainability of the lifestyle, business practice or industry to which the carbon footprint applies. There are many other factors, and aspects such as these drive an individual value structure.

Human consumers may wish to apply their value system to their everyday lives. Many aspects of this value system can be attributed to consumer choice. In a society the emphasizes personal liberties and individual volition, humans have many opportunities for decisions in everyday life that implement this value structure. Selections made by consumers of various goods, activities, services, expenditures and the like occur frequently in daily life. However, many people may not know or be aware of how these choices align with their value system. Take the example of routine grocery purchases. Many products result from a complex supply chain that involves product inception from natural resources, manufacturing and packaging, distribution, and retail sales. Various retail consumables span a similar product distribution chain, from manufacturing, sales, distribution and retail purchase and delivery. Each step in the sequence may affect various environmental, economic, societal or governmental aspects. For example, plant-based products are grown, harvested and shipped. An individual consumer may inquire about the natural harvested plant resource and location, what pesticides and chemicals were involved, and how far was it shipped. Manufactured goods may raise questions about natural resources consumed for minerals or plastics, the environmental impact of manufacturing, and the human labor force involved.

Configurations herein are based, in part, on the observation that modern consumables often rely on a complex mass production approach. Products and services directed to retail and consumer levels follow a well-established infrastructure network for correlating supply and need of such consumables. These consumables (food, merchandise, services) are often sought by consumers who have a personal value structure that reflects views of environmental, economic, health, safety and similar preferences. Unfortunately, conventional approaches to the delivery of consumables suffer from the shortcoming that it can be difficult to assess whether a selection of such consumables aligns with the value structure of the consumer. For example, in a grocery context, a consumer or purchaser may be presented with several options for a particular commodity item. It can be difficult to identify which of the available products is harvested in a sustainable manner, is free from harmful additives or pesticides, or does not derive from an oppressive labor practice.

While many people may be sensitive or concerned with such far reaching and complex issues, it is difficult to efficiently assess in a supermarket or other retail setting where many items are purchased in a relatively short time. Even reading a list of ingredients for each purchased product is cumbersome, let alone considering farther-reaching implications of the relevant supply chain. It would be beneficial if a resource existed where questions of how particular products align with an individual value structure could be addressed. Accordingly, configurations herein substantially overcome the shortcomings of conventional approaches by establishing a knowledge base of attributes about a set of available consumables, and presenting an analysis of ratings based on qualifying criteria for aspects such as nutrition, processing, environment and safety. Such an analysis is readily accessible via a rendering device such as a smartphone or similar personal device, and allows entry of a specific personal value structure for comparison with consumables under consideration.

In general, the qualifying criteria are each representative of an aspect of wellness of the user, of general human health, or of societal, economic or environmental norms that derive from an individual value structure, and preferences that are relevant to these individual values. Any suitable number of qualifying criteria may be employed, for example animal welfare and human rights.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a decision support environment suitable for use with configurations herein;

FIG. 2 is a data flow diagram of a decision support system in the environment of FIG. 1;

FIG. 3 is a flowchart of operation in the decision support system of FIGS. 1 and 2;

FIG. 4 is an example of ingredients processing logic for commodities supported by the decision support system of FIGS. 1-3;

FIG. 5 shows natural language logic for analyzing the ingredients of FIG. 4;

FIG. 6 shows the logic for scoring qualifying criteria for nutrition based on the commodities of FIG. 4;

FIG. 7 shows the logic for scoring qualifying criteria for industrial processing of the commodities of FIG. 4;

FIG. 8 shows the logic for scoring a qualifying criteria pertaining to environmental aspects of the commodities of FIG. 4;

FIG. 9 shows the logic for scoring a toxicity resulting from the commodities of FIG. 4; and

FIG. 10 shows an aggregation of qualifying criteria into a final score for comparison in the decision support system of FIGS. 1-9.

### DETAILED DESCRIPTION

In the discussion that follows, an example of a decision support system including a knowledge base, recommendation engine and mobile device GUI provides access to a coalesced arrangement of products (items) and related attributes for supporting analytics on a vast number of commodities including grocery items generally available for purchase and consumptions. Items supported by the disclosed decision support system may include any kind of commodity, grocery, beverage, consumable or other consumer-oriented item directed to a retail environment, and capable of description according to the attributes as disclosed herein. Qualifying criteria that represent important features or properties are analyzed, scored, and rendered for comparison with an individual value structure and priority of the user. The analyzed and rendered attributes are gathered and coalesced from various sources that would be intractable to research in real-time in a typical grocery purchase environment, therefore providing a specialized body of knowledge in an easily accessible form for consumer use. Any suitable commodity may be employed with corresponding qualifying criteria for facilitating the purchase decision.

Conventional approaches to providing web-based grocery assistance for selection, purchase and/or delivery have several shortcomings. Typically, product information for contemplated purchases is missing or lacking, for example by providing only basic nutrient information from a label, but without further analysis or details regarding sourcing, allergens, restrictive diets, etc. Others may perform a data rendering based on the SKU (Stock Keeping Unit, or bar code) label, which provides more information but falls short of analytics or sourcing data combined with a matching of preferences of individual users.

It would be beneficial to provide an artificial intelligence(AI) or machine learning (ML) solution that has the ability to enable value-based grocery shopping, through its personalized and seamless experience, wherein the value is presented and assessed beyond mere recitation of the partial information on the package or label.

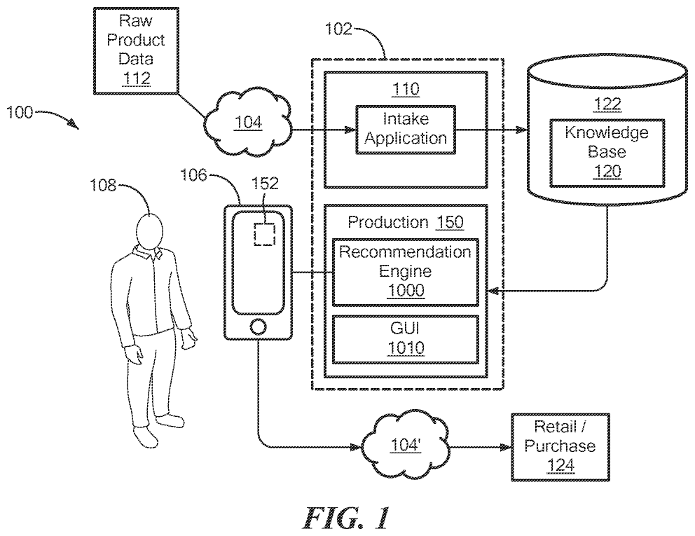

FIG. 1 is a context diagram of a decision support environment suitable for use with configurations herein. In the environment 100, a decision support system 102 includes an intake application 110 and a production application 150. The intake application 110 invokes a public access network 104 for gathering raw product data 112 about a number of products, services or items for storing in a knowledge base 120. The knowledge base 120 is supported on a repository 122 which may include any appropriate configuration of available storage servers, disk/SSD and/or cloud-based storage. The production application 150 supports a mobile app 152 launchable on any suitable device 106 for user 108 engagement.

The knowledge base 120 is responsive to a recommendation engine 1000 in the production application 150 for providing product data, recommendations, comparisons and analysis via a GUI (graphical user interface) 1010 in communication with the mobile app 152. A typical scenario involves consideration for purchase, either in person or internet/electronic/GUI based, or other consideration for consumption, such as research on items, products or commodities outside a direct retail context. User requests for specific items available through the production application may be fulfilled by a retail/purchase entity 124 by network 104′ invocation from the mobile app 152. The mobile app 152 enables users to perform values-based grocery shopping that conforms with their dietary and other preferences. This is achieved by evaluating food products on diet, health and sustainability factors using Natural Language Processing (NLP) and text mining logic for generating quantifiable scores, discussed further below.

In the example configuration, using grocery and food items as the available products, the shopping experience is personalized using the recommendation engine and a statistical model that uses inputs provided by the users regarding their diet and value preferences. The model learns from the users' online browsing patterns to suggest better-aligned alternatives for food products. Another variant of the aforementioned model uses NLP to generate an automated personalized shopping list, that accepts open text entry from the user, and automatically populates the shopping list with the best option, based on an individual's preferences and priorities related to diet, health, sustainability, and price. The decision support system 100 is also extrapolated to generate automated shopping carts, based on a user's past shopping cart distribution, or simply from input/scans of external shopping receipts.

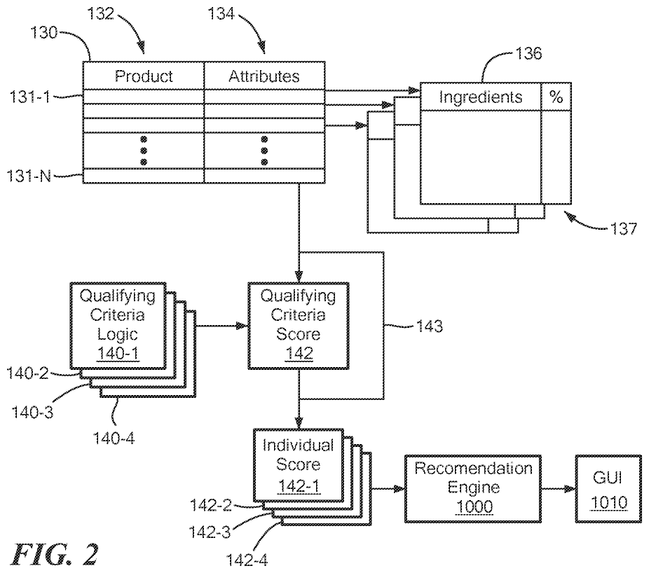

FIG. 2 is a data flow diagram of the decision support system 102 in the environment of FIG. 1. Referring to FIGS. 1 and 2, the knowledge base 120 includes a set of items or products 132, and corresponding attributes 134 in a plurality of entries 131-1 . . . 131-N (131 generally). The attributes 134 include tags of qualitative features, values for quantitative features, and generally include an ingredients list 136 for each product entry 131. The value-based recommendations generally include one or more qualifying criteria, each represented by scores reflective of how the product 132 meets the values, and also how the scores align with user 108 preferences for the values. The production application 150 includes qualifying criteria logic 140-1 . . . 140-4 (140 generally) for a plurality of qualifying criteria. Four are employed in the examples herein, however any suitable number may be applied. The qualifying criteria logic 140 generates a qualifying criteria score 142 for each of the applicable qualifying criteria, generally iterating as shown by arrow 143 for each of the qualifying criteria logic 140 for each of the products 132.

The example configuration determines whether a user has indicated a preference for specifying the magnitude of qualifying criteria, and if so, receives from the user, the magnitude of qualifying criteria for comparison with the computed qualifying criteria. Generally, the qualifying criteria includes at least one qualifying criteria score 142, such that each qualifying criteria score is indicative of a scalar value. Equal weighting may be employed if the user has not specified any preference. The qualifying criteria score is representative of an aspect of wellness and based on attributes of the item having a relevant bearing on the respective qualifying criteria score.

The result of the qualifying criteria logic 140 is an individual score 142-1 . . . 142-4 (142 generally) for each of the qualifying criteria. The qualifying criteria logic 140 allows rendering, for a plurality of qualifying criteria, a respective qualifying criteria score 142 for a plurality of items, and renders, for each item of the plurality of items, an overall aggregated score based on each qualifying criteria score. The GUI 1010 may then arrange renderings of each item of the plurality of items based on a matching correspondence with the magnitude of qualifying criteria. In the example arrangement, this would include displaying and ranking a list or tiled selection screen of the products available for purchase, and/or with a circular graph segment illustrating each of the individual scores 142-N.

Briefly, the qualifying criteria discussed in the examples below include nutrition, processing, environment, and food safety. Additional criteria, such as animal welfare, human rights and others could be established based on the value system employed and the products, items or services used. These examples illustrate the use of the individual scores 142 by the recommendation engine 1000 for rendering recommendations and comparisons to the user 108 via the GUI 1010.

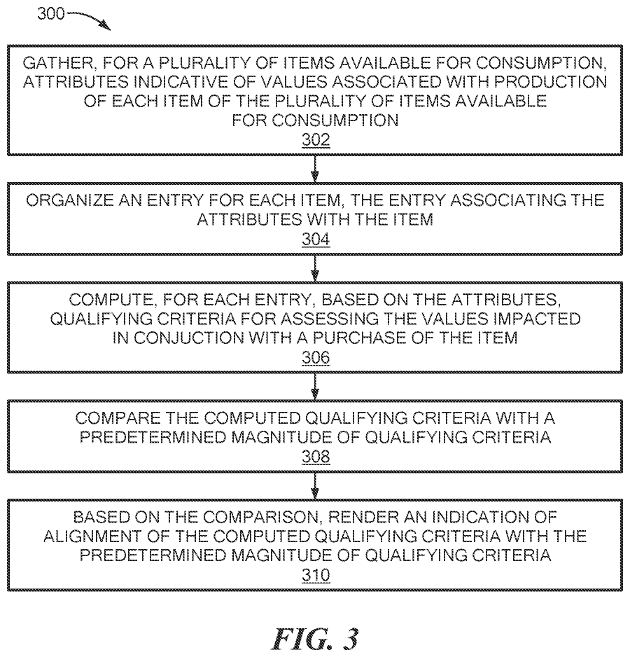

FIG. 3 is a flowchart of general operation in the decision support system of FIGS. 1 and 2. In the decision support environment 100 having a plurality of consumable commodity items or products 132 available to users 108 having preferences for weighing values associated with consumed resources in view of a personal value structure of the user, a method for coalescing and rendering a quantitative value rating apportioned to an item includes gathering attributes 134 for each of a plurality of items available for consumption, as depicted at step 302. A robust knowledge base may cover several hundred thousand items, such that the attributes 134 are indicative of values associated with the production of each item of the plurality of items available for consumption. The attributes 134 are based on SKU level data from web sites of private grocers, as well as publicly available databases like USDA (United States Department of Agriculture). The attributes contemplate any cause, effect or impact that the item or product imposes on the qualifying criteria in placing the product or item into the stream of commerce. Production of the item, as evaluated by the qualifying criteria, includes the ingredients, manufacture and transport of the item, as well as any subsequent effect such as disposal of the item or packaging. The features acquired include product descriptions, such as name, ingredient list, nutritional information, as well as pricing and store availability information. The collected data is then cleaned and aggregated using a product matching algorithm, which consolidates information from multiple sources, and keeps the best and most complete product information, ensuring the use of salient data for analysis. The intake application 110 organizes an entry 131 for each product 132 or item, such that the entry associates the attributes 134 with the item, as shown at step 304.

Redundant and duplicative information is filtered by the product matching algorithm for determining the same products acquired from different data sources and mainly happens via proximity analysis of various descriptive features of products, which is significant in instances where the universal product identifier, e.g., SKU/UPC code is missing. Text-based features such as name, ingredients and the package size are analyzed for proximity using Levenshtein distance, and numerical features such as stated nutritional information may be directly matched. The product matching algorithm is useful to enable pricing comparison functionality and acts as an automated quality assurance system, which only keeps the best quality data from multiple sources.

Attributes are further derived and normalized from the raw product data 112 by an NLP-based multi-class classification algorithm that uses text mining to determine the target class to which a product belongs. Each class (referred to as a tag), contains a corpus of words to mine for in the ingredient list and name of the product. The presence of such words determines to which class the product belongs. There can be multiple classes to which a product can belong. For example, a product can be both Gluten-Free and Vegan.

K-Means Clustering and Set Theory are used to auto-assign certain classes that are correlated with other tags, in order to reduce computation time and improve interpretability. An example would be a cluster containing all vegan-friendly classes, like ‘Vegan’, ‘No Eggs’, ‘No Milk’. This is achieved by using a Word2Vec approach, in which all the word corpora are assigned numerical vectors, and their proximity analysis is done to determine various relevant clusters, that are leveraged to execute K-Means clustering to assign correlated tags to products.

Once the knowledge base 120 is populated, the production application 150 may be invoked to compute, based on the attributes, qualifying criteria for each entry 131 for assessing the values impacted in conjunction with a purchase of the item, as disclosed at step 306. The populated knowledge base 120 then contains information related to each of the qualifying criteria to allow computation of the individual score 142 for that qualifying criteria. For the attributes and qualifying criteria discussed herein, it is apparent that the ingredients list 136 is useful in computing a qualifying criteria for nutrition. However, other more attenuated value assessments are also provided. Each ingredient emanates from a particular supply chain, and often a geographic region. Attributes pertaining to harvesting methods and geographic location may also be relevant. Some ingredients point to artificial processing by chemical or mechanical means. These and other aspects are codified and compared by the qualifying criteria logic 140.

The recommendation engine 1000 compares the computed qualifying criteria reflected by the score 142 with a predetermined magnitude of qualifying criteria, which may be based on user values or predetermined generic criteria, as depicted at step 308. Based on the comparison, the recommendation engine identifies the item or items that best align with the predetermined magnitude of qualifying criteria, typically the user's values. The GUI renders an indication of alignment of the computed qualifying criteria with the predetermined magnitude, as shown at step 310. This rendering may simply be the top aligning or several highest aligning items, simply identified by name or image. It may also render scoring and/or alignment information, and may further indicate a degree of a matching correspondence (e.g. such as XX % match with the user's qualifying criteria). In other words, the numeric operations and raw values and scores need not be rendered to the users, as some users may simply rely on the recommendation engine to respond with product recommendations, knowing that they are based on the qualifying criteria already entered by the user.

It should be emphasized that the notion of user “values” refers to the individual (or default) preferences for personal objectives, ethical beliefs and socio-economic desires. The quantitative “scores” embodied in the qualifying criteria scores are a necessarily numeric value representation in a scalar assessment for each of the qualifying criteria.

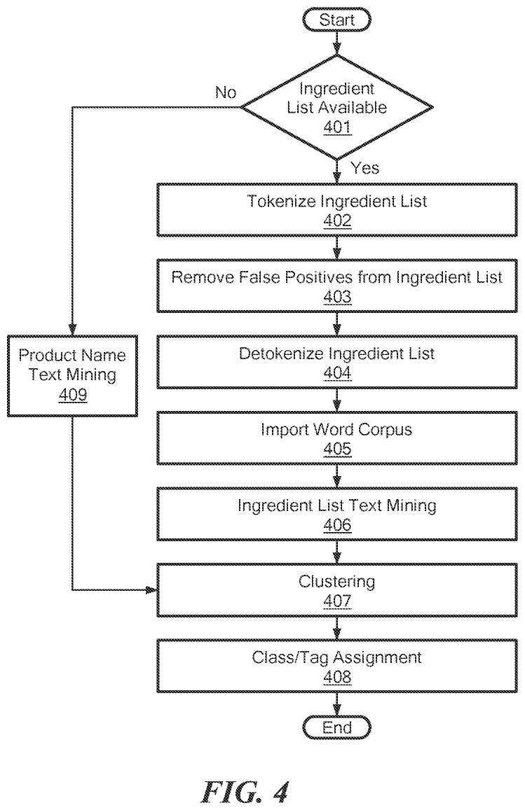

FIG. 4 is an example of ingredients processing logic for commodities supported by the decision support system of FIGS. 1-3. The intake application 110 generates the attributes 134 from the raw product data 112.

Referring to FIGS. 1-4, the process starts by checking the availability of an ingredient list for the product, as depicted in step 401. If available, the feature is tokenized based on commas (“,”), to separate out individual ingredients, as shown at step 402. An analysis of false positives then takes place, to remove words such as ‘Gluten’, ‘Dairy’, in case a product states gluten-free or dairy-free, as well as instances such as ‘Milk’, ‘Butter’ etc., in cases where the product is plant based, as depicted at step 403. This is done to ensure products are not classified as having allergens such as lactose, milk etc. The ingredient list is then detokenized to obtain a normal text feature, as disclosed at step 404, after which word corpus for classification is imported, shown at step 405. The word corpus contains group of words that belong to target classes:

WC_{vegan}={w₁,w₂,w₃, . . . wₙ}

Here, the set of n words mean all the ingredients that, if found present in the ingredient list, indicate that a given product cannot be consumed by people following a vegan diet.

The next step text mines the ingredient list for the presence of words belonging to different corpora, as depicted at step 406, after which a clustering procedure is initiated, shown at step 407, which aims at grouping similar correlated groups into one single group, as explained below:

C_{vegan}⊂C_{vegetarian}

Cₖₒₛₕₑᵣ⊆C_{shellfish}

C_{vegan}⊂Cₖₒₛₕₑᵣ

The sample equations show all the products not fit for vegan consumption belong to the broader cluster of the products not fit for vegetarian consumption. Similarly, all the products having shellfish cannot be consumed by people with kosher dietary preferences, etc. Alternatively, in the case where the ingredient list is absent, the product name is text mined 409, and steps 407, 408 remain the same. A final step 408 is a class assignment, in which, after cluster analysis and text mining, classes are assigned to products, as given below:

pᵢ={vegan,vegetarian,kosher,shellfish}

This means a given product pᵢ cannot be consumed by people with Vegan, Vegetarian, or Kosher dietary preference, or who have a shellfish allergy.

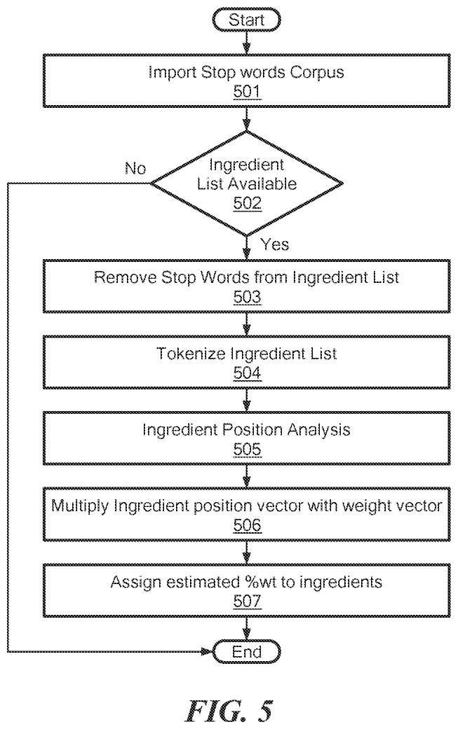

An ingredient weight estimator, shown in FIG. 5, to analyze the distribution of ingredients (by % wt), as the information is typically not provided by food manufacturers in the USA, but is a significant input for the approach herein. The general version uses a predetermined weight vector and analyzes the positional distribution of ingredients to assign a weight by volume value to each ingredient. The logic followed by the aforementioned approach relies on the FDA standard, which states: “An ingredient list on a food label, as defined by the FDA, is the listing of each ingredient in descending order of predominance.” This rationale applies most accurately to solid food products, such as dry grains/dry pasta, or other boxed foods. Alternate configurations adjust the weight vector according to the type of product the system is analyzing. This is generally used for products that have more water content in them, such as beverages and pre-cooked canned items.

FIG. 5 shows natural language logic for analyzing the ingredients of FIG. 4. Intake of the ingredients 136 is particularly significant for the qualifying criteria, as much of the logic 140 draws from these attributes. Referring to FIGS. 1-5, gathering attributes further includes, for each item or product 142 of the plurality of items, traversing the public access network 104 for ingredient information about the item. The intake application 110 gathers, for each item, a list of ingredients contained in the item. Since ingredients are listed in decreasing order, the intake application estimates, from the list of ingredients, the percentage composition of each ingredient in the list of ingredients. The list of ingredients 136 and the percentage composition 137 corresponding to each ingredient are then stored.

Gathering of data by the intake application 110 may take a variety of forms, including automated crawlers, keyword searching and the like to retrieve relevant information. In a particular configuration, for each item in the plurality of items, the intake application traverses a public access network for information about the item, and determines, based on the information, the availability of qualitative class data about ingredients in the item. Based on the determined availability, the intake application 110 extracts, from the information, an attribute indicative of a predetermined dietary pattern that the item is consistent with. Also, if available, the intake application 110 extracts attributes indicative of allergens contained in the item, and stores the extracted attributes 134 in the knowledge base 120.

Various approaches to gathering, processing and matching the attributes of the products based on NLP approaches using corpora of gathered words pertaining to a particular attribute. Accordingly, configurations herein validate the gathered attributes by accumulating the information from a plurality of sources on the public access network for each item. The disclosed approach retrieves, from each source of the plurality of sources, information pertaining to respective attributes and correlates the retrieved information for determining a match for the respective attribute from the respective sources.

In FIG. 5, the sequence starts with importing the stop word corpus at step 501, in which we aim to remove all the additives in a product that, we know from research, do not contribute significantly to the overall % wt of a given product. An example of such a trace ingredient would be Monosodium Glutamate. After confirming the availability of the ingredient list at step 502, after which the stop words are removed from the ingredient list (step 503) the positional order of the ingredients remains, at step 504. After tokenizing the cleaned form of ingredients, the ingredient position analysis takes place, depicted at step 505, resulting in a positional vector, for example:

[I₁:1,I₂:2, . . . Iₙ:n]

### Where:

I₁, I₂ . . . ::Exact ingredient names, and their position in the ingredient list and 1≤n≤6
   Values from Table I are invoked to multiply the ingredient position vector with the ingredient vector, as depicted at step 506, based on the listing of each ingredient being in descending order of predominance.

TABLE I

| Number of | 1. Ingredient | 2. Ingredient | 3. Ingredient | 4. Ingredient | 5. Ingredient | 6. Ingredient |
|---|---|---|---|---|---|---|
| Ingredients | Weight | Weight | Weight | Weight | Weight | Weight |
| 1 | 100% | | | | | |
| 2 | 80% | 20% | | | | |
| 3 | 80% | 16% | 4% | | | |
| 4 | 80% | 16% | 3.2% | 0.8% | | |
| 5 | 80% | 16% | 3.2% | 0.64% | 0.16% | |
| 6 | 80% | 16% | 3.2% | 0.64% | 0.128% | 0.032% |

For example, assuming n=4, the ingredient weight vector will be given as follows:

IW=[0.8,0.16,0.032,0.008]

The respective percentages given in Table 1 are multiplied by a vector of the ordered ingredients, following removal of minutiae or trace contributors at step 503, to approximate a percentage of each ingredient. Table 1 codifies estimates for the first 6 ingredients; other approximations and number of ingredients may be represented.

Having populated the knowledge base with attributes 134 including the ingredients 136 and percentages 137 from the raw product data 112, the recommendation engine is ready to coalesce the data and compute the qualifying criteria scores 142.

In conjunction with the GUI, and based on the comparison, the production application 150 renders an indication of alignment of the computed qualifying criteria with the predetermined magnitude of qualifying criteria. A score value is generated for each of the qualifying criteria and averaged or aggregated to arrive at an overall score, colloquially referred to as a “GreenScore” approximating an overall rating across all qualifying criteria. Users 108 may also have preferences as to weighting scores based on the different qualifying criteria.

As indicated above, informing and matching the users of the qualifying criteria associated with their contemplated items, products or other commodities and includes several qualifying criteria from which to consider and compare. The following examples employ nutrition, processing, environment and safety, however any suitable number of qualifying criteria could be employed.

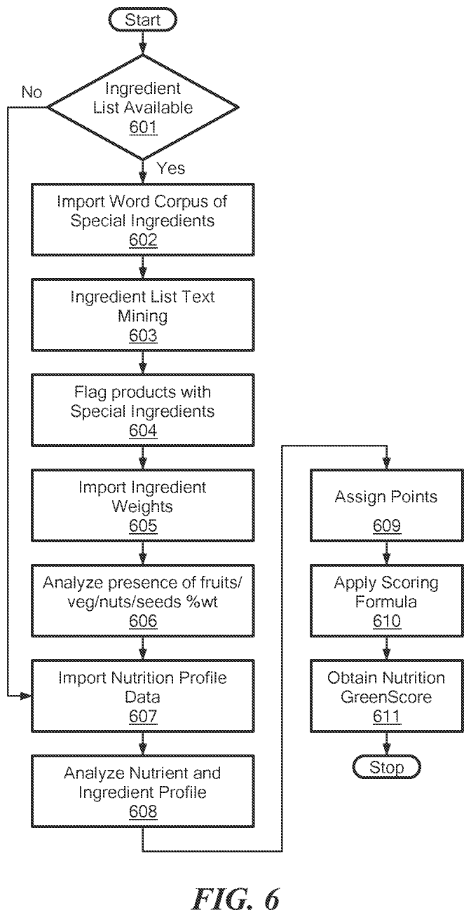

FIG. 6 shows the logic for scoring a qualifying criteria for nutrition based on the commodities of FIG. 4. Referring to FIGS. 1-6, the qualifying criteria score 140-1 for nutrition includes a profiling system that classifies and ranks food and beverage products by their nutrient and ingredient composition, on a per-serving basis. The algorithm analyzes the Nutritional Label information as well as the Ingredients list to assign points based on ‘Nutrients to Encourage’, and ‘Nutrients to Limit’, and the final points obtained are translated into a scale of 0-100, which makes for the qualifying criteria nutrition score 142-1. Certain “beyond the label information,” that the general population is not aware of, is determined by executing text mining techniques on the Ingredient List, to determine factors, such as ‘Sugarnyms’, or different types of added sugars, isolated sources of proteins, extracted sources of fibers, the estimated amount of Omega-3 fatty acids, etc.

Computing the qualifying criteria score 140-1 for nutrition further comprising comparing, for each item, the list of ingredients in the item to a set of preferred nutrition entries and a set of disfavored nutrition entries. In other words, some ingredients increase the nutrition score and others decrease it. The qualifying criteria logic 140-1 compares each ingredient in the list of ingredients to the set of preferred nutrition entries, and to the set of disfavored nutrition entries. Based on the comparison, the logic computes a nutrition score, based on each ingredient having a match in the set of preferred nutrition entries or the disfavored nutrition entries, either yielding a quantity of the respective ingredient.

In further detail, commencing at step 601, a check is performed to determine if an ingredients list is available, and if so, importing a corpus or list of special ingredients at step 602. This is similar to the ingredients intake of FIG. 5, however it involves a subset of ingredients from which nutrition is derived. Ingredients 136 of the knowledge base 120 are mined to identify special ingredients, as depicted at step 603, based on a match, and products with special ingredients flagged, at step 604. For the products so flagged, the corresponding ingredient weights as computed above are retrieved, as disclosed at step 605. Particular percentages of fruits, vegetables, nuts and seeds are analyzed at step 606. These types of food are generally higher in nutrition and endure less processing. Nutrition profile data other than the ingredients list is imported at step 607, and also for products 132 for which ingredients lists were unavailable.

All information for high nutrition ingredients, lower nutrition ingredients, special ingredients such as allergens, and general nutrition profile has now been gathered, and the nutrient and profile information is analyzed, as depicted at step 608. Points are assigned (or deducted) for healthy and unhealthy aspects of ingredients, as disclosed at step 609, and a scoring formula applied at step 610. The coalesced score for the nutrition qualifying criteria 142-1 is obtained as step 611, and is invoked as discussed further below.

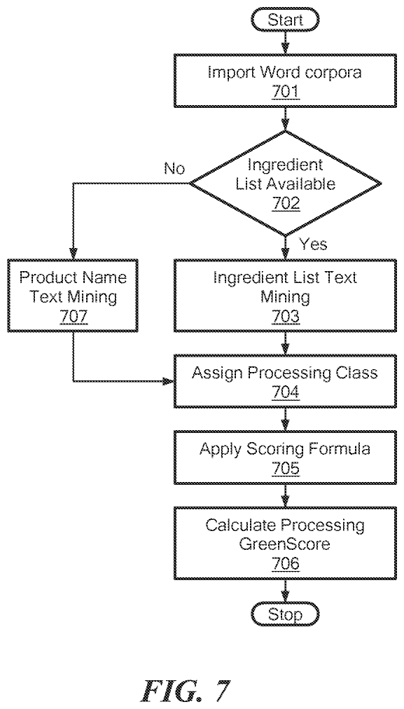

FIG. 7 shows the logic for scoring qualifying criteria for industrial processing of the commodities of FIG. 4. The qualifying criteria score 142-2 for processing assesses the physical, biological and chemical alterations that food products undergo to change food structure, nutritional content, texture, taste, and shelf-life. In addition, the scoring system takes into account the addition of food additives, with a focus on purpose, source, and processing method (i.e. artificial/man-made).

The algorithm is an NLP-based classification model, that contains a corpus of words to mine in the ingredient list and name of the product. The presence of such words determines to which class the product belongs, out of three exemplary target classes of Minimally Processed, Moderately Processed and Highly Processed. A linear model is also defined, that is used to assign a Processing Score, from a scale of 0-100, based on parameters determined by the kind of words in the defined corpora that are present in a given product, and their correlation to negative health and the level of processing.

In the example arrangement, gathering the attributes further comprises receiving a corpus of words indicative of a processing class, as depicted at step 701. This corpus of words is based on the likelihood of alterations made to the respective item. Generally, processing entails either physical/mechanical processing, as in chopping, slicing and/or grinding; chemical, such as emulsifiers, thickeners, etc., and preservatives, nutritional, such as adding nutrients and vitamins, and thermal, typically heating, freezing or cooking to a prepared state (as in foods that only need defrosting or reheating).

The ingredients list is referenced, at step 702, and if found, a natural language search and text mining is performed for a match of words descriptive of the item with the corpus of words indicative of processing, as depicted at step 703. If there is no ingredients list, text mining is performed based on the product name alone, as disclosed in step 707. The qualifying criteria logic 140-2 generates, based on the match of words, a designated processing class of the respective item and a processing score indicative of a magnitude of processing. This includes, based on a match with the mined text, designating processing actions operable on an item of the plurality of items. In other words, identifying what processing is appliable to the product in question. The qualifying criteria logic 140-2 identifies, for each processing action, which of the processing classes are affected as a result of applying the identified processing action. It then identifies, for each item if the plurality of items, processing actions performed, and correlates the processing action performed to a mapping of processing actions to processing classes affected. Processing actions include steps such as freezing, marinating, carbonating, canning and frying, to name several. Each of these actions triggers one or more of the processing classes of physical, chemical, nutritional and thermal. In other words, some words may be indicative of mechanical processing, others of chemical processing. The qualifying criteria logic 140-2 may also designate, for each of the processing actions, a health factor indicative of a relative impact of the processing action, and compute, based on the number of mapped processing classes and the health factor, a processing score for the item, such as a point for each class triggered by a processing action. A scoring formula applies for each determined processing step or action, as depicted at step 705. The logic 140-2 then computes the processing score based 142-2 on a count of the number of mapped processing classes affected from the processing action on the item, as disclosed at step 706.

Another consideration is provided by external entities that implement or enforce preparation certifications and standards, such as USDA grading of meat. In such cases, the natural language search based on the ingredients may indicate a certification applied to the item by a recognized entity, and supersede the generated processing score based on alterations implied by the certification.

Images pertaining to official certifications often accompany the certification, appearing as a logo or trademark of the issuing entity. Accordingly, in particular configurations, identification of a certification may occur by performing image recognition on graphical images returned by the public access network pertaining to the item, and comparing the graphical image to images indicative of certification by the recognized entity. If a match with an image indicative of certification is found, this supersedes the generated processing score based on alterations implied by the certification.

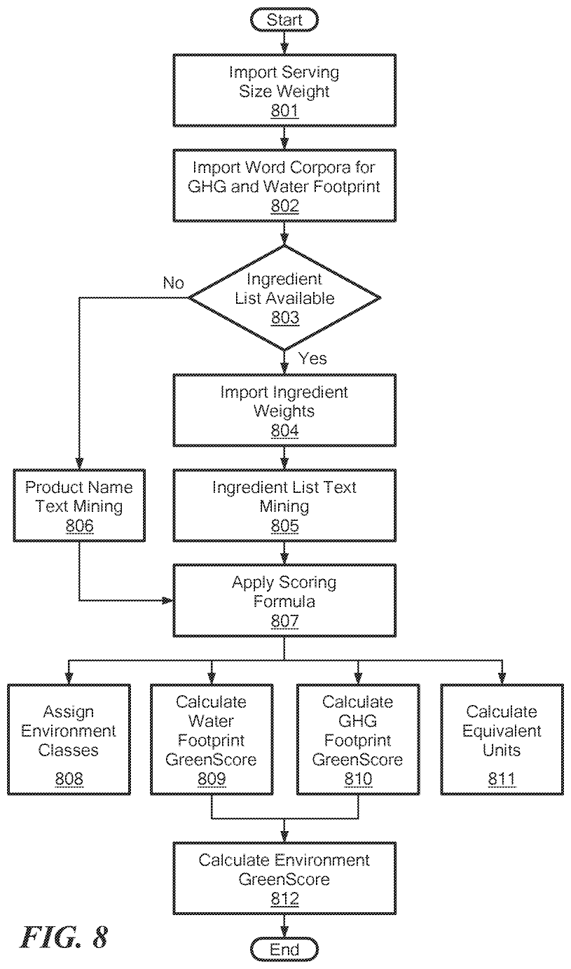

FIG. 8 shows the logic for scoring qualifying criteria pertaining to environmental aspects of the commodities of FIG. 4. Qualifying criteria logic 140-3 for environment includes the environmental impact of supply, providing, growing or other supply chain impacts resulting from the inclusion of a particular ingredient. A qualifying criterion for an environment estimates a product's water footprint in gallons and its greenhouse gas footprint in carbon dioxide equivalents, on a per-serving basis.

This is achieved using linear equations, that analyze the ingredient list of the products, the estimated % weight of each ingredient, and the water consumption and greenhouse gas emissions linked to each ingredient within the product. In cases where the serving size of a product is unknown, assumptions are made based on the type of product the system is processing. Based on the final number estimated, the products are assigned an environment score, from a scale of 0-100, both in carbon and water footprints, and are classified into one of six possible classes (very low, low, moderate, high and very high carbon/water footprint). The information is also converted into user-friendly equivalent units, such as ‘Running a shower for x minutes’ and ‘Diving a car for x miles.’ Referring to FIGS. 1-4 and 8, at step 801, importing a serving size weight for a product. It should be apparent that an environmental impact from production scales proportionately with the quantity of the product and its constituent ingredients. A corpus of words pertaining to greenhouse gases (GHG) and water footprint is imported, as depicted at step 802. A check is performed for an available ingredients list, as shown in step 803. Based on availability of ingredients, the previously calculated percentage weight of ingredients is imported, as depicted in step 804. Ingredient list text mining is performed based on the corpora of words, as disclosed at step 805. Alternatively, if no ingredient attributes are available, text mining based on the product name is invoked for determining included ingredients, as depicted in step 806. In either case, scoring based on the quantity of each ingredient and the GHG and water footprint effect, as depicted at step 807.

The environment score may also favor or be limited to a carbon footprint and carbon-based ramifications of the items and the production and supply chain thereof.

From the scoring, environmental classes from very low to very high are generated, based on the aggregate score of all ingredients, as shown at step 808. These environmental classes may be rendered later in specific queries for each product.

The qualifying criteria logic 140-3 determines, for each ingredient, environmental ratings including a quantity-based water consumption factor and a quantity-based greenhouse gas emission factor, such that each of the environmental ratings scales linearly based on the quantity. Alternate units for quantity may also be converted, as depicted in step 811. Recalling that processing of the ingredients as in FIGS. 4 and 5 has generated percentage attributes of each ingredient, the qualifying criteria environmental logic 140-3 computes, for each ingredient in each item, a quantity; and for each item, computes an environmental score based on the greenhouse gas emission factor (step 810) and the water consumption factor (step 809) aggregated for the computed quantity of each ingredient. Overall environmental qualifying criteria score 142-3 is computed, as depicted at step 812.

Another qualifying criteria score involves receiving a set of public health factors relevant to ingredients of food consumables, such that each of the public health factors denotes an ingredient and a quantity deemed to be harmful. An item is identified as having at least one of the ingredients denoted by the received public health factors, and a health score is computed based on the percentage composition of the identified ingredients of each respective identified item of the plurality of items.

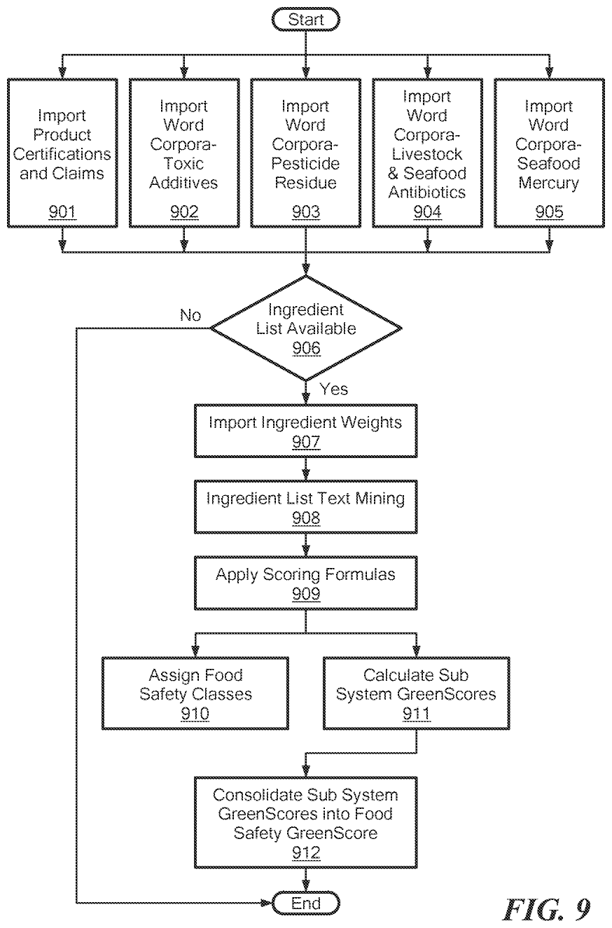

FIG. 9 shows the logic for scoring toxicity resulting from the commodities of FIG. 4. Qualifying criteria logic for food safety 140-4 generates a score that is a combination of various factors that are related to the Public Health concerns of food products, such as the presence of (i) toxic/carcinogenic food additives, (ii) pesticide/herbicide residue, (iii) antibiotics used to raise livestock and seafood, and (iv) mercury accumulation in seafood. The logic 140-4 receives a set of public health factors relevant to ingredients of food consumables, such that each of the public health factors denotes an ingredient and a quantity deemed to be harmful, and identifies each item of the plurality of items having at least one of the ingredients denoted by the received public health factors.

For the safety score, a number of keyword and text mining steps are invoked, some of which overlap with attributes gathered previously. Product certifications and claims are imported, as depicted in step 901. Certifications might assuage concerns about the manner in which ingredients were caught or harvested. A corpora of toxic additives are imported, as depicted in step 902. These are used to compare to the ingredients, and generally reduce the safety score. A pesticide/residue corpus is imported, as depicted in step 903, to evaluate harmful chemical residues that may remain on certain ingredients or whole fruits/vegetables. An antibiotic usage corpus is imported at step 904, and heavy/cumulative/residual metals corpora are imported, as depicted at step 905, for comparison with foods that may contain mercury or other harmful substances that propagate the food chain.

A check is performed, at step 906, to determine if an ingredients list is available for a particular product. If so, the ingredient weights (percentages) are imported, as depicted at step 907, and text mining performed based on the imported corpora of steps 901-905 for identifying food safety issues in the ingredients, as disclosed at step 908. A scoring formula codified in the qualifying criteria logic for food safety 142-4 is applied, as depicted at step 909, and resulting safety classes for each product assigned at step 910. Concurrently, subsystem green scores are calculated, corresponding to the comparisons based on steps 901-905, as disclosed at step 911. Each of the subsystem factors of steps 901-905 has a separate algorithm, and use NLP based classifiers to determine the presence of certain ingredients using text mining, and the linear equations that are defined for each of the sub-systems are used to assign a final aggregated Food Safety Score on a scale of 0-100, that is a weighted average of the Sub-System Scores, with higher weight given to the sub-systems that score the lowest. Based on the individual ingredients, an overall health score 142-4 is computed based on the percentage composition of ingredients for each respective identified item of the plurality of items.

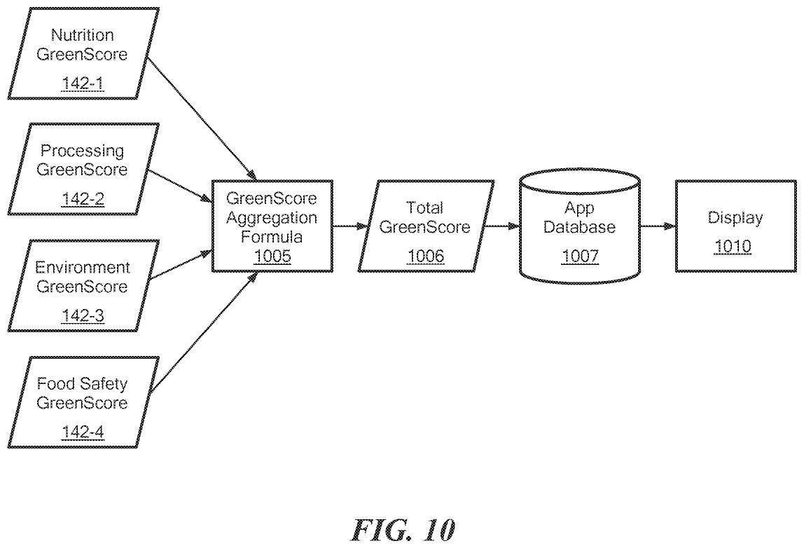

Following computation of the applicable qualifying criteria scores 142, a cumulative or aggregate score may be computed. FIG. 10 shows an aggregation of qualifying criteria into a final score for comparison in the decision support system of FIGS. 1-9. A final score 1006 is assigned on a product (SKU) level on a scale of 0-100. This may be a simple average of the four scores Nutrition, Processing, Environment, and Food Safety, or may be weighted by an aggregation formula 1005 for different emphasis. An application database 1007 incorporates the final score 1006 in conjunction with each the attributes for each product 132, along with the constituent scores 142 and other attributes. The GUI 1010 renders the scores 142 and other attributes upon user request.

Alternate configurations and arrangements invoke the product 132 and attribute offerings to generate real-time shopping lists and carts by automatically populating the best matching products in the cart. An example would be if someone types ‘milk’, the recommendation engine will automatically add the best matching product in the cart, hence eliminating the need to manually search on the mobile application 152.

The recommendation engine 1000 may also be used to suggest related products to the user, based on the product currently being rendered on the GUI 1010. A group of products that pair well with the given product is displayed to the user, and the same can be directly added to the cart, e.g. if a user is looking at dry pasta, then a marinara sauce, and a dry herb that matches best with user preferences is suggested to the user, and the entire bundle can be added to the cart and purchased. A feedback loop is used to determine the success rate of such add-to-cart events, and the system adjusts its recommendation accordingly.

Further configurations may apply the logic of the recommendation engine to a history of previous purchases, and compute aggregate scores of the qualifying criteria over time, as well as suggesting alternative for increasing the scores for customers that strive to improve either their overall performance or in one specific area, such as environmental.

In particular configurations, the approach gathers attributes for a plurality of items based on historical purchases, and computes the qualifying criteria for each item of the plurality of items based on historical purchases in conjunction with the gathered attributes of the current plurality of items available for consumption. It then computes an aggregate for each of the qualifying criteria, and computes an overall summation indicative of each of the qualifying criteria. This may further include computing an aggregate carbon footprint score based on a greenhouse gases component of the qualifying criteria.

Alternatives for improving the relevant scores may also be suggested. A report renders, for each of the qualifying criteria, and for each of the attributes defining the respective qualifying criteria, an aggregation of the attributes based on the gathered attributes of the current plurality of items available for consumption. The approach then renders an aggregation of the gathered attributes for a plurality of items based on historical purchases. The same report also renders an indication of a substitute item based on the rendered item and yielding an improvement in the attributes of the item. This yields a similar item with an improved score in one or more of the qualifying criteria.

The process starts with getting the user purchase data from either their past receipt submissions, or store loyalty card integrations. The purchased products are then linked to products in the database, and key product metrics are extracted, as given below:

1. All four qualifying criteria scores (Nutrition, Processing, Food Safety, Environment)

2. Water Footprint (In Gallons) per package size

3. Carbon Footprint (In CO2 equivalents) per package size

4. Binary value indicating if a product is high in saturated fat

5. Binary value indicating if a product has toxic additives

The purchased cart is then analyzed to calculate two main factors:

A personal score based on the individual scores of the qualifying criteria. This value is calculated by calculating the weighted average of the total value of the qualifying criteria scores for all the past products purchased by a user, effectively indicating a users overall consumer healthfulness and sustainability. This value is helpful for the users to keep a track of how they are improving over time, plus acts as a point of relative comparison with other individuals. A community average score based on an average number calculated for all the purchases of app users in their region, to help a user determine how he/she is doing as compared to the other users.

The system also shows a breakdown of the user scores into the 4 main verticals of nutrition, food safety, environment and processing, to give detailed information on how a user is performing in the 4 qualifying criteria, as well as a comparison of the same with other users of the app. For every purchase made by a user, a critical ‘impact metric’ is computed to quantify and track the health statistics and sustainability impacts of a users' grocery purchases/food consumption. This includes users' estimated water footprint, carbon footprint, amount of products with high saturated fats, and amount of products with added sugars per grocery shopping trip made by the user.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state-drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor-controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. In a decision support environment having a plurality of consumable commodity items available to users having preferences for weighing values associated with consumed resources in view of a personal value structure of the user, a method for coalescing and rendering a quantitative value rating apportioned to an item, comprising:
gathering, for a plurality of items available for consumption, attributes indicative of values associated with production of each item of the plurality of items available for consumption;
organizing an entry for each item of the plurality of items, the entry associating the attributes with the item;
computing, for each entry, based on the attributes, qualifying criteria for assessing the values impacted in conjunction with a purchase of the item;
comparing the computed qualifying criteria with a predetermined magnitude of qualifying criteria; and
rendering an item contemplated for consumption, based on the comparison indicating an alignment of the computed qualifying criteria with the predetermined magnitude of qualifying criteria.

2. The method of claim 1 further comprising rendering, along with the item contemplated for consumption, an indication of the computed qualifying criteria.

3. The method of claim 1 further comprising determining whether a user has indicated a preference for specifying the predetermined magnitude of qualifying criteria, and if so, receiving, from the user, the magnitude of qualifying criteria for comparison with the computed qualifying criteria.

4. The method of claim 1 further comprising inferring the predetermined magnitude of qualifying criteria for comparison with the computed qualifying criteria of an item, based on a user.

5. The method of claim 1 wherein the qualifying criteria includes at least one qualifying criteria score, each qualifying criteria score indicative of a scalar value, the qualifying criteria score representative of an aspect including at least one of health, sustainability and welfare, and based on the attributes of the item having a relevant bearing on the respective qualifying criteria score.

6. The method of claim 1 further comprising rendering an indication of alignment of the qualifying criteria score with the predetermined magnitude of qualifying criteria.

7. The method of claim 5 further comprising:
rendering, for a plurality of qualifying criteria, a respective qualifying criteria score for a plurality of items, the plurality of items being contemplated for consumption;
rendering, for each item of the plurality of items, an overall aggregated score based on each qualifying criteria score of the plurality of qualifying criteria scores;
arranging renderings of each item of the plurality of items based on a matching correspondence of the overall aggregated score with the magnitude of qualifying criteria; and
rendering an indication of a degree of the matching correspondence of each item of the plurality of items.

8. The method of claim 1 wherein gathering attributes further comprises:
for each item of the plurality of items, traversing a public access network for ingredient information about the item;
gathering, from the ingredient information, for each item, a list of ingredients contained in the item;
estimating, from the list of ingredients, a percentage composition of each ingredient in the list of ingredients; and
storing, in a database of items, the list of ingredients and the percentage composition corresponding to each ingredient.

9. The method of claim 1 further comprising:
for each item in the plurality of items, traversing a public access network for information about the item;
determining, based on the information, an availability of qualitative class data about ingredients in the item;
based on the determined availability, extracting, from the information, an attribute indicative of a predetermined dietary pattern that the item is consistent with;
based on the determined availability, extracting, from the information, an attribute indicative of allergens contained in the item; and
storing, in a database of items, a set of the extracted attributes.

10. The method of claim 8 further comprising comparing, for each item, the list of ingredients in the item to a set of preferred nutrition entries and a set of disfavored nutrition entries;
comparing each ingredient in the list of ingredients to the set of preferred nutrition entries;
comparing each ingredient in the list of ingredients to the set of disfavored nutrition entries; and
based on the comparison, computing a nutrition score, computing further based on for each ingredient having a match in the set of preferred nutrition entries or the disfavored nutrition entries, a quantity of the respective ingredient.

11. The method of claim 1 wherein gathering the attributes further comprises:
receiving a corpus of words, the corpus of words indicative of a processing class, the corpus of words based on a likelihood of alterations made to the respective item;
for each item of the plurality of items available for consumption, performing a natural language search on a public access network for a match of words descriptive of the item with the corpus of words; and
generating, based on the match of words, a designated processing class of the respective item and a processing score indicative of a magnitude of processing.

12. The method of claim 11 further comprising:
designating processing actions operable on an item of the plurality of items;
identifying, for each processing action, which of the processing classes are affected as a result of applying the identified processing action;
identifying, for each item if the plurality of items, processing actions performed;
correlating the processing action performed to a mapping of processing actions to processing classes affected; and
adjusting the processing score based on a count of number of mapped processing classes affected from the processing action on the item.

13. The method of claim 12 further comprising:
designating, for each of the processing actions, a health factor indicative of a relative impact of the processing action; and
computing, based on the number of mapped processing classes and the health factor, a processing score for the item.

14. The method of claim 11 further comprising:
determining if the natural language search indicated a certification applied to the item by a recognized entity; and
superseding the generated processing score based on alterations implied by the certification.

15. The method of claim 8 wherein gathering the attributes further comprises:
for each ingredient, determining environmental ratings including a quantity based water consumption factor and a quantity based greenhouse gas emission factor, each of the environmental ratings scaling linearly based on the quantity;
computing, for each ingredient in each item, a quantity; and
for each item, computing an environmental score based on the greenhouse gas emission factor and the water consumption factor aggregated for the computed quantity of each ingredient.

16. The method of claim 8 wherein gathering the attributes further comprises:
for each ingredient, determining environmental ratings including a quantity based greenhouse gas emission factor;
computing, for each ingredient in each item, a quantity; and
for each item, computing an environmental score based on the greenhouse gas emission factor aggregated for the computed quantity of each ingredient.

17. The method of claim 8 wherein gathering the attributes further comprises:
receiving a set of public health factors relevant to ingredients of food consumables, each of the public health factors denoting an ingredient and a quantity deemed to be harmful;
identifying each item of the plurality of items having at least one of the ingredients denoted by the received public health factors; and
computing, for the identified items, a health score based on the percentage composition of each respective identified item of the plurality of items.

18. The method of claim 14 further comprising:
performing an image recognition on graphical images returned by the public access network pertaining to the item;
comparing the graphical image to images indicative of certification by the recognized entity; and
if a match with an image indicative of certification is found, superseding the generated processing score based on alterations implied by the certification.

19. The method of claim 1 further comprising validating the gathered attributes by accumulating the information from a plurality of sources on the public access network for each item,
retrieving, from each source of the plurality of source, information pertaining to a respective attribute; and
correlating the retrieved information for determining a match for the respective attribute from the respective sources.

20. A computing device for gathering, analyzing and rendering commodity recommendations based on attributes and comparisons with qualifying criteria reflective of a value structure, comprising:
a network interface configured to gather attributes indicative of values associated with production of each item of the plurality of items available for consumption;
a knowledge base to store and organize an entry for each item, the entry associating the attributes with the item;
logic to compute, for each entry, based on the attributes, qualifying criteria for assessing the values impacted in conjunction with a purchase of the item;
a recommendation engine configured to compare the computed qualifying criteria with a predetermined magnitude of qualifying criteria; and
an interface to a graphical user interface (GUI) to, based on the comparison, render an indication of alignment of the computed qualifying criteria with the predetermined magnitude of qualifying criteria.

21. The device of claim 20 wherein the qualifying criteria includes at least one qualifying criteria score, each qualifying criteria score indicative of a scalar value, the qualifying criteria score representative of an aspect of wellness and based on attributes of the item having a relevant bearing on the respective qualifying criteria score.

22. The device of claim 20 wherein the GUI is further operable to
render, for a plurality of qualifying criteria, a respective qualifying criteria score for a plurality of items;
render, for each item of the plurality of items, an overall aggregated score based on each qualifying criteria score of the plurality of qualifying criteria scores; and
arrange renderings of each item of the plurality of items based on a matching correspondence with the magnitude of qualifying criteria.

23. The device of claim 20 further comprising an intake application configured to:
for each item of the plurality of items, traverse a public access network for ingredient information about the item;
import, from the ingredient information, for each item, a list of ingredients contained in the item;
estimate, from the list of ingredients, a percentage composition of each ingredient in the list of ingredients; and
store, in a database of items, the list of ingredients and the percentage composition corresponding to each ingredient.

24. The method of claim 1 further comprising:
gathering attributes for a plurality of items based on historical purchases;
computing the qualifying criteria for each item of the plurality of items based on historical purchases in conjunction with the gathered attributes of the current plurality of items available for consumption;
computing an aggregate for each of the qualifying criteria; and
computing an overall summation indicative of each of the qualifying criteria.

25. The method of claim 24 further comprising:
computing an aggregate carbon footprint score based on a greenhouse gases component of the qualifying criteria.

26. The method of claim 24 further comprising:
for each of the qualifying criteria,
for each of the attributes defining the respective qualifying criteria;
rendering an aggregation of the attributes based on the gathered attributes of the current plurality of items available for consumption; and
rendering an aggregation of the gathered attributes for a plurality of items based on historical purchases;
rendering, for at least one of the items, an indication of a substitute item, the substitute item based on the rendered item and yielding an improvement in the attributes of the item.

27. A computer program embodying program code on a non-transitory medium that, when executed by a processor, performs a method for, in a decision support environment having a plurality of consumable commodity items available to users having preferences for consumed resources, coalescing and rendering attributes related to an item, the method comprising:
gathering, for a plurality of items available for consumption, attributes indicative of resources expended in producing each item of the plurality of items available for consumption;
organizing an entry for each item, the entry associating the attributes with the item;
computing, for each entry, based on the attributes, qualifying criteria for assessing the resources expended in conjunction with a purchase of the item; and
rendering the qualifying criteria along with the qualifying criteria for at least one other item of the plurality of items.
